(19) United States
(12) Patent Application Publication
(10) Pub. No.: US 2006/0228406 A1
(43) Pub. Date: Oct. 12, 2006

(54) Transfection reagent for non-adherent suspension cells

(75) Inventors: Henry Chi-Shon Chiou, Encinitas, CA (US); Charles G. Shevlin, San Diego, CA (US)

Correspondence Address: GREENLEE WINNER AND SULLIVAN P C, 4875 PEARL EAST CIRCLE, SUITE 200, BOULDER, CO, 80301, US

(73) Assignee: Invitrogen Corporation, Carlsbad, CA (US)

(21) Appl. No.: 11/384,178

(22) Filed: Mar. 17, 2006

Related U.S. Application Data
(60) Provisional application No. 60/663,309, filed on Mar. 17, 2005.

(51) Int. Cl. A61K 48/00; A61K 9/127

(52) U.S. Cl. 424/450; 514/44

(57) ABSTRACT

The present invention discloses liposomal transfection reagents for delivery of macromolecules and other compounds into cells, particularly non-adherent suspension cells. They are especially useful for the DNA-dependent transformation of cells. Methods for their preparation and use as intracellular delivery agents are also disclosed.

### CROSS-REFERENCE TO RELATED APPLICATIONS

This application claims priority under 35 U.S.C. 119(e) to U.S. provisional application Ser. No. 60/663,309, filed Mar. 17, 2005, which is incorporated by reference in its entirety herein.

### BACKGROUND OF THE INVENTION

Lipid aggregates such as liposomes have been found to be useful as delivery agents for introducing macromolecules, such as DNA, RNA, proteins, and small chemical compounds, such as pharmaceuticals, to cells. In particular, lipid aggregates comprising cationic lipid components have been shown to be especially effective for delivering anionic molecules to cells. In part, the effectiveness of cationic lipids is thought to result from enhanced affinity for cells, many of which bear a net negative charge. Also in part, the net positive charge on lipid aggregates comprising a cationic lipid enables the aggregate to bind polyanions, such as nucleic acids. Lipid aggregates containing DNA are known to be effective agents for efficient transfection of target cells.

The structure of lipid aggregates varies, depending on composition and method of forming the aggregate. Such aggregates include liposomes, unilamellar vesicles, multilamellar vesicles, micelles and the like, having particle sizes in the nanometer to micrometer range. Methods of making lipid aggregates are well known in the art. The main drawback to use of conventional phospholipid-containing liposomes for delivery is that the material to be delivered must be encapsulated and the liposomal composition has a net negative charge that is not attracted to the negatively charged cell surface. By combining cationic lipid compounds with a phospholipid, positively charged vesicles and other types of lipid aggregates can bind DNA, which is negatively charged, can be taken up by target cells, and can transfect target cells. (Feigner, P. L. et al. (1987) Proc. Natl. Acad. Sci. USA 84:7413-7417; Eppstein, D. et al., U.S. Pat. No. 4,897,355.)

Delivery of nucleic acids such as DNA into cells is important, in part, because these nucleic acids can encode the necessary information to direct the cells to express an important recombinant protein. Recombinant proteins, such as recombinant enzymes and recombinant antibodies, have many practical uses in society, including uses as food additives, in diagnostic tests, and as pharmaceutical agents. Furthermore, methods using recombinant proteins help scientists to better understand life and improve our ability to identify new medicines and diagnostics. Therefore, improved methods for transfection and recombinant protein product can result in improved foods, medicines, and diagnostics.

Methods for incorporating cationic lipids into lipid aggregates are well-known in the art. Representative methods are disclosed by Felgner et al., supra; Eppstein et al. supra; Behr et al. supra; Bangham, A. et al. (1965) M. Mol. Biol. 23:238-252; Olson, F. et al. (1979) Biochim. Biophys. Acta 557:9-23; Szoka, F. et al. (1978) Proc. Natl. Acad. Sci. USA 75:4194-4198; Mayhew, E. et al. (1984) Biochim. Biophys. Acta 775:169-175; Kim, S. et al. (1983) Biochim. Biophys. Acta 728:339-348; and Fukunaga, M. et al. (1984) Endocrinol. 115:757-761. Commonly used techniques for preparing lipid aggregates of appropriate size for use as delivery vehicles include sonication and freeze-thaw plus extrusion. See, e.g., Mayer, L. et al. (1986) Biochim. Biophys. Acta 858:161-168. Microfluidization is used when consistently small (50-200 nm) and relatively uniform aggregates are desired (Mayhew, E., supra). Aggregates ranging from about 50 nm to about 200 nm diameter are preferred; however, both larger and smaller sized aggregates are functional.

Current transfection reagents, however, are not adequately effective for non-adherent, suspension cell cultures, which can be excellent cell cultures for recombinant protein production, and can be toxic to cells adapted to non-adherent growth. Transfection reagents that exhibit high transfection efficiency in adherent cell cultures have greatly reduced transfection efficiency in non-adherent suspension cells and some will kill over half of the treated cell population. Alternative reagents and formulations are needed that are non-toxic and effective for the transfection of non-adherent, suspension cell cultures, especially for protein production.

### SUMMARY OF THE INVENTION

The present invention provides liposomal transfection reagents that achieve high transfection efficiency in cells, including, but not limited to, cells that have been adapted for growth as non-adherent suspension cell cultures. The transfection reagents of the present invention comprise a composition of at least one cationic lipid and at least one neutral lipid. The cationic lipid-neutral lipid composition is formed into liposomes. The resulting liposomes are polycationic, able to form stable complexes with anionic macromolecules, including, but not limited to, nucleic acids or proteins. The polyanion-lipid complex interacts with cells making the polyanionic macromolecule available for absorption and uptake by the cell. The liposomal transfection reagents of the present invention are less toxic to certain cells, including, but not limited to, non-adherent cells, and provide higher transfection efficiency than transfection reagents known in the art. The present invention also provides expression kits comprising liposomal transfection reagents and methods for transfecting cells using such reagents.

In one embodiment of the invention, the transfection reagents comprise a liposomal composition comprising one or more cationic lipids, or mixtures thereof, where the cationic lipids have the formula: 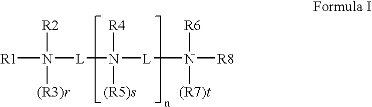
or salts or polycations thereof, where r, s and t independent of one other are 0 or 1 to indicate the presence or absence of the individual group, wherein when N is tetravalent it is positively charged, and wherein at least one of r or t is 1 or at least one s is 1 (the lipid carries at least one positive charge);
where L is a divalent organic radical independently selected from the group consisting of a C₁₋₁₀ alkylene group, wherein one or more non-neighboring —CH₂— groups can be replaced with an O or S atom, and wherein one or more carbons of the alkyl group can be substituted with an OH, SH, SR or OR group where R is an alkyl group having from 1 to about 6 carbon atoms;
where n is an integer from 0 to 10;
where R1-R8 are independently selected from the group consisting of hydrogen and a C₁₋₂₂ alkyl, C₂₋₂₂ alkenyl, C₂₋₂₂ alkynyl and C₆₋₂₂ aryl, optionally substituted with one or more of an alcohol, aminoalcohol, hydroxyl, amine, carbohydrate, ether, polyether, amide, polyamide, ester, mercaptan, urea, thiourea, heterocyclic group, or heterocyclic aromatic group and/or wherein one or more non-neighboring —CH₂— groups can be replaced with an O or S atom;
where at least two of R1-R8 are independently selected from the group consisting of a C₈₋₂₂ alkyl, C₈₋₂₂ alkenyl, C₈₋₂₂ alkynyl and C₈₋₂₂ aryl, optionally substituted with one or more of an alcohol, aminoalcohol, hydroxyl, amine, carbohydrate, ether, polyether, amide, polyamide, ester, mercaptan, urea, thiourea, heterocyclic group, or heterocyclic aromatic group and/or one or more non-neighboring —CH₂— groups can be replaced with an O or S atom; and
where at least two of R1-R8 are independently selected from the group consisting of a C₁₋₆ alkyl, C₂₋₆ alkenyl and C₂₋₆ alkynyl.

In more specific embodiments, of formula I when n is 0, none of R1-R8 is an alkyl group substituted with an aminoalcohol and all other variables are as defined above. In another more specific embodiment, of formula I, when n is 1 all of R1-R3 and R6-R8 are groups other than hydrogen and all other variables are as defined above.

In more specific embodiments of formula I, all of r, s and t are 1 and all N carry a positive charge. Dependent upon the value of n, there may be multiple R5 groups in the cationic lipid of formula I. Each R5, independent of other R5s in the lipid, can be selected from any of the various chemical groups defined above. Further, any one or more of the multiple R5 groups may be present (i.e., the value of s for that R5 group is 1) so that the N to which it is attached is positively charged, or any one or more of the multiple R5 groups may be absent (i.e., the value of s for that R5 group is 0) so that the N to which it is attached is not positively charged. The lipids of formula I can include cations having a total of 1 to n+2 positive charges on N in the lipid. The lipids of formula I include those having a total of n+2 positive charges on N in the lipid.

The anions employed in the formation of salts formula I include, but are not limited to, halides, sulfate, carboxylates, acetates, phosphate, nitrate, trifluoroacetate, glycolate, pyruvate, oxalate, malate, succinicate, fumarate, tartarate, citrate, benzoate, methanesulfonate, ethanesulfonate, p-toluenesulfonate, salicylate and the like.

Preferably, at least two of R1-R8 are independently selected from the group consisting of a C₁₂₋₂₀ alkyl, C₁₂₋₂₀ alkenyl, C₁₂₋₂₀ alkynyl and C₁₂₋₂₀ aryl, optionally substituted with one or more of an alcohol, aminoalcohol, hydroxyl, amine, carbohydrate, ether, polyether, amide, polyamide, ester, mercaptan, urea, thiourea, heterocyclic group, or heterocyclic aromatic group. More preferably, at least two of R1-R8 are a C₁₆₋₁₈ alkyl. More preferably, at least four of R1-R8 are a C₁₆₋₁₈ alkyl.

It is also preferable that at least two of R1-R8 are independently selected from the group consisting of a C₁₋₆ alkyl. More preferably, at least two of R1-R8 are CH₃. More preferably, at least four of R1-R8 are CH₃.

It is preferable that n is 2. It is also preferable that one R group attached to each nitrogen atom designated in Formula I is a C₁₆₋₁₈ alkyl, and one R group attached to each nitrogen atom designated in Formula I is CH₃.

In a further embodiment, R1 and R8 are independently selected from the group consisting of a hydrogen and a C₁₋₈ alkyl, C₂₋₈ alkenyl, C₂₋₈ alkynyl and C₂₋₈ aryl, optionally substituted by one or more of an alcohol, aminoalcohol, hydroxyl, amine, carbohydrate, ether, polyether, amide, polyamide, ester, mercaptan, urea, thiourea, heterocyclic group, or heterocyclic aromatic group;

R2, R4, and R6 are independently selected from the group consisting of hydrogen and a C₁₋₂₂ alkyl, C₂₋₂₂ alkenyl, C₂₋₂₂ alkynyl and C₁₋₂₂ aryl, optionally substituted with one or more of an alcohol, amine, amide, ether, polyether, polyamide, ester, mercaptan, urea, or thiol; and

R3, R5 and R7 are independently selected from the group consisting of hydrogen and a C₁₋₆ alkyl.

Specific embodiments of this invention include cationic lipids of formula I where n is an integer from 0 to about 2; R1 and R8 are independently selected from the group consisting of a C₁₋₆ alkyl optionally substituted by one or more of an alcohol, aminoalcohol, amine, ether, amide, ester, mercaptan, urea or thiourea; R3 and R7 are independently selected from the group consisting of hydrogen and CH₃; R2, R4 and R6 are independently selected from the group consisting of a C₁₂₋₂₀ alkyl; and R5 is selected from the group consisting of hydrogen and a C₁₋₆ alkyl. Specific embodiments include those in which none of R1-R8 are groups substituted with alcohol, aminoalcohol, or amine groups.

Further embodiments of this invention include cationic lipids of formula I where n is 2; R1 and R8 are CH₃; R3 and R7 are hydrogen; R2, R4 and R6 are an alkyl having 16, 17, or 18 carbon atoms; and R5 is CH₃.

In another embodiment of the invention, the transfection reagents have a liposomal composition comprising one or more cationic lipids, or mixtures thereof, where the cationic lipids have the formula: 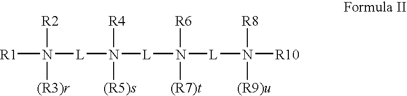
or salts and polycations thereof, where r, s, t and u independent of one other are 0 or 1 to indicate the presence or absence of the individual group, wherein when N is tetravalent it is positively charged, and wherein at least one of r, s, t or u is 1;
where L is a divalent organic radical independently selected from the group consisting of an alkylene group having from 1 to about 10 carbon atoms, wherein one or more non-neighboring —CH₂— groups can be replaced with an O or S atom, and wherein one or more carbon atoms of the group can be substituted with an OH, SH, SR or OR group, where R is an alkyl group having from 1 to about 6 carbon atoms;
where R1-R10 are independently selected from the group consisting of hydrogen and a C₁₋₂₂ alkyl, C₂₋₂₂ alkenyl, C₂₋₂₂ alkynyl and C₆₋₂₂ aryl, optionally substituted with one or more of an alcohol, aminoalcohol, hydroxyl, amine, carbohydrate, ether, polyether, amide, polyamide, ester, mercaptan, urea, thiourea, heterocyclic group, or heterocyclic aromatic group;
where between 2 and 4 groups of R1-R10 are independently selected from the group consisting of a C₈₋₂₂ alkyl, C₈₋₂₂ alkenyl, C₈₋₂₂ alkynyl and C₆₋₂₂ aryl, optionally substituted with one or more of an alcohol, aminoalcohol, hydroxyl, amine, carbohydrate, ether, polyether, amide, polyamide, ester, mercaptan, urea, thiourea, heterocyclic group, or heterocyclic aromatic group; and where between 0 and 6 groups of R1-R10 are independently selected from the group consisting of a C₁₋₆ alkyl, C₂₋₆ alkenyl and C₂₋₆ alkynyl.

In specific embodiments, of formula II, each of R1, R2, R3, R8, R9 and R10 are groups other than hydrogen and the other variables in the formula are as defined above. The anions employed in the formation of salts of formula II include, but are not limited to, halides, sulfate, carboxylates, acetates, phosphate, nitrate, trifluoroacetate, glycolate, pyruvate, oxalate, malate, succinicate, fumarate, tartarate, citrate, benzoate, methanesulfonate, ethanesulfonate, p-toluenesulfonate, salicylate and the like.

It is to be understood that if one of the 2 to 4 groups selected from the group consisting of a C₈₋₂₂ alkyl, C₈₋₂₂ alkenyl, C₈₋₂₂ alkynyl and C₈₋₂₂ aryl is R3, R5, R7 or R9, then r, s, t and u, respectively, must equal 1. Similarly, if one of the 0 to 6 groups selected from the group consisting of a C₁ alkyl, C₂₋₆ alkenyl and C₂₋₆ alkynyl is R3, R5, R7 or R9, then r, s, t and u, respectively, must be 1.

Preferably, between 2 and 4 groups of R1-R10 are selected from the group consisting of a C₁₂₋₂₀ alkyl, C₁₂₋₂₀ alkenyl, C₁₂₋₂₀ alkynyl and C₁₂₋₂₀ aryl, optionally substituted with one or more of an alcohol, aminoalcohol, hydroxyl, amine, carbohydrate, ether, polyether, amide, polyamide, ester, mercaptan, urea, thiourea, heterocyclic group, or heterocyclic aromatic group. More preferably, between 2 and 4 groups of R1-R10 are a C₁₆₋₁₈ alkyl. More preferably, R2, R4, R6 and R8 are a C₁₆₋₁₈ alkyl.

Preferably, between 2 and 6 groups, more preferably between 4 and 6 groups, of R1-R10 are independently selected from the group consisting of a C₁₋₆ alkyl, C₂₋₆ alkenyl and C₂₋₆ alkynyl. More preferably, between 4 and 6 groups of R1-R10 are CH₃. More preferably, R3, R5, R7 and R9 are CH₃.

Preferably between 2 to 6 of R1-R10 are selected from C₁₋₆ alkyl, and between 2 to 4 of R1-R10 are selected from C₈₋₂₂ alkyl (more preferably C₁₂₋₂₀ alkyl), and any remaining R1-R10 groups are hydrogen or are absent.

One embodiment of the present invention includes cationic lipids of formula II where all of r, s, t and u are 1. In other embodiments, at least three of r, s, t, or u are 1. In other embodiments, at least two of r, s, t, or u are 1. In other embodiments, at least one of r, s, t, or u are 1.

One embodiment of the present invention includes cationic lipids of formula II where R1 and R10 independently are selected from the group consisting of an alkyl having from about 1 to about 22 carbon atoms; where R2, R3, R5, R7, R8 and R9 independently are selected from the group consisting of hydrogen and an alkyl having from 1 to about 6 carbon atoms; and where R4 and R6 independently are selected from the group consisting of an alkyl, alkenyl and alkynyl having from 2 to about 22 carbon atoms wherein one or more non-neighboring —CH₂— groups can be replaced with an O or S atom.

Specific embodiments of this invention include cationic lipids of formula II where R1, R3, R9 and R10 are selected from the group consisting of hydrogen and an alkyl having 1 to about 3 carbon atoms; R2, R4, R6 and R8 are selected from the group consisting of an alkyl having about 12 to about 20 carbon atoms, and R5 and R7 are CH₃.

Further embodiments of this invention include cationic lipids of formula II where L is selected from the group consisting of an alkylene having 2 to about 4 carbon atoms; R1, R5, R7 and R10 are CH₃; R3 and R9 are hydrogen; and R2, R4, R6 and R8 are an alkyl having 16 carbon atoms.

As used herein, the term “divalent organic radical” refers to a chemical linker or moiety that forms two bonds to different portions of a molecule. The two bonds of the linker or moiety may be on the same atom, or may be on two different atoms. Preferably, the two bonds are on two different atoms that are on opposite ends of the linker or moiety.

The transfection reagents of the present invention further comprise one or more neutral lipids. Neutral lipids useful in the composition of the transfection reagents include, but are not limited to, DOPE, 1,2-dioleoyl-sn-glycerol-3-phosphocholine (DOPC), cholesterol and mixtures thereof.

One embodiment of the present invention comprises transfection reagents comprising one or more cationic lipids of formula I and one or more neutral lipids. More specifically, the transfection reagents comprise one or more cationic lipids of formula I and DOPE, or cholesterol or mixtures thereof.

One embodiment of the present invention comprises transfection reagents comprising one or more cationic lipids of formula II and one or more neutral lipids. More specifically, the transfection reagents comprise one or more cationic lipids of formula II and DOPE, or cholesterol or mixtures thereof.

In one embodiment of the present invention, the molar ratio of the one or more cationic lipids to the one or more neutral lipids in the transfection reagents is between 1:0.8 and 1:3.0. In further embodiment, the molar ratio of the one or more cationic lipids to the one or more neutral lipids in the transfection reagents is between 1:1.6 and 1:2.3. In further embodiment, the molar ratio of the one or more cationic lipids to the one or more neutral lipids in the transfection reagents is between 1:1.6 and 1:1.9.

In another embodiment of the present invention, the molar ratio of the one or more cationic lipids to the one or more neutral lipids in the transfection reagents is between about 1:1.5 and 1:1.7. In another embodiment, the molar ratio of the one or more cationic lipids to the one or more neutral lipids in the transfection reagents is 1:1.5 or above.

The transfection reagents of the present invention optionally include additional helper lipids, such as 2,3-dioleyloxy-N-[2(sperminecarboxamido)ethyl]-N,N-dimethyl-1-propanaminium trifluoroacetate (DOSPA), 1,2-dioleoyl-sn-glycerol-3-(phospho-L-serine) (DOPS), DOGS, DOTMA, or mixtures thereof, as well as the cationic lipids of formula I and formula II and the one or more neutral lipids.

The transfection reagents of the present invention are formed into liposomes and mixed with the macromolecules to be introduced into the cell. Macromolecules that can be delivered to cells with the transfection reagents according to the present invention are macromolecules having at least one negative charge in the molecule. Such macromolecules include, but are not limited to, proteins, polypeptides and nucleic acids, such as RNA and DNA.

Methods of forming liposomes are well known in the art and include, but are not limited to, sonication, extrusion, extended vortexing, reverse evaporation, and homogenization, which includes microfulidization.

Sonication typically produces small, unilamellar vesicles (SUV) with diameters in the range of 15-50 nm. Bath sonicators are the most widely used instrumentation for preparation of SUV (Avanti Polar Lipids, Inc., 700 Industrial Park Drive, Alabaster, Ala. 35007). Sonication is accomplished by placing a test tube containing the suspension in a bath sonicator (or placing the tip of a sonicator in the test tube) and sonicating for 5-10 minutes above the gel-liquid crystal transition temperature of the lipid. Mean size and uniformity is influenced by lipid composition and concentration, temperature, sonication time, power, volume, and sonicator tuning. Reverse evaporation is used to form larger liposome vesicles (>1000 nm) known as giant unilamellar vesicles (GUV's).

Another method of forming liposomal compositions is extrusion. Lipid extrusion is a technique in which a lipid suspension is forced through a polycarbonate filter with a defined pore size to yield particles having a diameter near the pore size of the filter used. Extrusion through filters with pores having an approximately 100 nm diameter typically yields large, unilamellar vesicles (LUV) with a mean diameter of 120 nm-140 nm. Mean particle size also depends on lipid composition and is reproducible from batch to batch.

In one embodiment of the present invention, the formed liposomes are approximately 120 nm to 800 nm in diameter.

The present invention is useful to deliver macromolecules to cells, which include but are not limited to CHO, NIH3T3, HEK293-MSR, HeLa, A549, PC12, HepG2, Jurkat, U937, COS-7, Vero, BHK and ME-180 cell lines. The present invention is particularly useful for non-adherent suspension cells, including, but not limited to, suspension CHO cells, suspension BHK cells, suspension NS0 cells, suspension HeLa cells and suspension HEK293 cells.

The transfection methods of the present invention can be applied to in vitro and in vivo transfection of cells, particularly to transfection of eukaryotic cells including animal cells. The methods of this invention can be used to generate transfected cells which express useful gene products. The methods of this invention can also be employed as a step in the production of transgenic animals. The methods of this invention are useful as a step in any therapeutic method requiring introducing of nucleic acids into cells. In particular, these methods are useful in cancer treatment, in in vivo and ex vivo gene therapy, and in diagnostic methods. The transfection compositions of this invention can be employed as research reagents in any transfection of cells done for research purposes. Nucleic acids that can be transfected by the methods of this invention include DNA and RNA from any source comprising natural bases or non-natural bases, and include those encoding and capable of expressing therapeutic or otherwise useful proteins in cells, those which inhibit undesired expression of nucleic acids in cells, those which inhibit undesired enzymatic activity or activate desired enzymes, those which catalyze reactions (Ribozymes), and those which function in diagnostic assays.

The reagents and methods provided herein can are also readily adapted to introduce biologically active anionic macromolecules other than nucleic acids including, among others, polyamines, polyamine acids, polypeptides, proteins, biotin, and polysaccharides into cells. Other materials useful, for example as therapeutic agents, diagnostic materials and research reagents, can be complexed by the polycationic lipid aggregates and delivered into cells by the methods of this invention.

The methods and materials of the invention are useful in the development and practice of cell based assays and in the screening of libraries of molecules by cell based assays. In such assays, one or more cells are contacted with a test compound after the macromolecule, particularly an expression vector, is introduced into the one or more cells. Preferably, the one or more cells are contacted with the test compound for a selected time, for example within 5 days, after the macromolecule is introduced into the one or more cells.

This invention also includes kits including transfection kits which include one or more of the compounds of formulas I, II or mixtures thereof as liposomal compositions.

This invention also provides reagents and methods for transfecting a recombinant protein-expressing plasmid DNA into suspension cells in order to produce high amounts of that protein.

This invention also provides reagents and methods for transiently transfecting suspension cells with an appropriate reporter gene expression construct so as to produce a transiently expressing reporter cell suitable for a cell-based assay.

### BRIEF DESCRIPTION OF THE DRAWINGS

FIG. 1 is a table showing different transfection reagents administered at different concentrations to a culture of CHO suspension cells.

FIG. 2 is graph showing the toxicity and transfection efficiency of various transfection reagents including transfection reagents of the present invention.

### DETAILED DESCRIPTION OF THE INVENTION

The present invention provides polycationic lipid compounds used in combination with neutral lipids (e.g., DOPE, DOPC and cholesterol) and optionally helper lipids (e.g., DOGS, DOPS, DOTMA and DOSPA) to prepare liposomes suitable for transfection or delivery of compounds to target cells, either in vitro or in vivo.

The compounds of formula (I) and formula (II), described above, are cationic and thus form stable complexes with various anionic macromolecules, particularly polyanions, including, but not limited to, nucleic acids. These compounds associate with anions via their cationic portions. By using cationic charges, the anion-lipid complexes may be adsorbed on cell membranes, thereby facilitating uptake of the desired compound by the cells.

The transfection reagents of the present invention are mixed with the desired macromolecules to form a liposome-macromolecule complex, which is administered to target cells. The target cells are contacted with the liposome-macromolecule complex under conditions that permit the liposome-macromolecule complex to enter the cells. These conditions include sufficient contact time between the liposome-macromolecule complex and the cell, typically from 24 hrs up to 72 hrs, temperature, typically at about room temperature, and standard conditions for maintaining the cells or culture of cells as are known in the art. Those of ordinary skill in the art can readily optimize transfection conditions for a given transfection agent, macromolecule to be delivered and cell line to be transfected.

The methods and transfection regents of the present invention include formulations of cationic lipids and neutral lipids useful in delivering macromolecules to cells that are not receptive to transfection or delivery of macromolecules using current transfection reagents. Current transfection reagents such as Lipofectamine 2000™ exhibit high transfection efficiency in adherent CHO cultures, a transfection rate of about 50% to 80%. However, this reagent achieves only 7% to 10% transfection efficiency in suspension CHO cells and kills over half the treated cell population. Similarly, other transfection reagents Lipofectin™ and DMRIE-C have up to approximately 10% to 50% transfection efficiency in suspension CHO cells with cell death ranging from 20% to over 50%. Cellfectin™ exhibits a transfection efficiency of approximately 20% to 30% in suspension CHO cultures with a cytotoxicity of about 20% to 50%. The formulations of the present invention can achieve transfection efficiencies of 50% or greater in suspension CHO cells with toxicities of less than 20%.

This relatively high transfection efficiency and low toxicity result in improved efficiencies with respect to recombinant protein production and cell-based assays. For example, recombinant proteins can be produced using transiently transfected suspension cells, rather than transfected adherent cells, according to methods provided herein. The ability to use suspension cells, including, but not limited to, suspension CHO cells, for recombinant protein production allows for an easier and more efficient scale-up procedures, since these cells can be grown in large volumes using standard cell culturing methods for suspension cells. Furthermore, methods provided herein permit cell-based assays to be performed with suspension cultures that transiently express recombinant reporter genes, which is more efficient that performing similar assays on stably transfected cells and/or adherent cells.

Definitions

The following terms and phrases are defined herein as follows:

With respect to chemical formulas herein:

The term “alkyl” refers to a monoradical branched or unbranched saturated hydrocarbon chain preferably having from 1 to 22 carbon atoms and to cycloalkyl groups having one or more rings having 3 to 22 carbon atoms. Short alkyl groups are those having 1 to 6 carbon atoms including methyl, ethyl, propyl, isopropyl, butyl, isobutyl, tert-butyl, pentyl and hexyl groups, including all isomers thereof. Long alkyl groups are those having 8-22 carbon atoms and preferably those having 12-22 carbon atoms as well as those having 12-20 and those having 16-18 carbon atoms. The term “cycloalkyl” refers to cyclic alkyl groups of from 3 to 22 carbon atoms having a single cyclic ring or multiple condensed rings. Cycloalkyl groups include, by way of example, single ring structures such as cyclopropyl, cyclobutyl, cyclopentyl, cyclooctyl, and the like, or multiple ring structures such as adamantanyl, and the like.

The term “alkenyl” refers to a monoradical of a branched or unbranched unsaturated hydrocarbon group preferably having from 2 to 22 carbon atoms and to cycloalkenyl groups having one or more rings having 3 to 22 carbon atoms wherein at least one ring contains a double bond. Alkenyl groups may contain one or more double bonds (C═C) which may be conjugated. Preferred alkenyl groups are those having 1 or 2 double bonds. Short alkenyl groups are those having 2 to 6 carbon atoms including ethylene (vinyl) propylene, butylene, pentylene and hexylene groups, including all isomers thereof. Long alkenyl groups are those having 8-22 carbon atoms and preferably those having 12-22 carbon atoms as well as those having 12-20 carbon atoms and those having 16-18 carbon atoms. The term “cycloalkenyl” refers to cyclic alkenyl groups of from 3 to 22 carbon atoms having a single cyclic ring or multiple condensed rings in which at least one ring contains a double bond (C═C). Cycloalkenyl groups include, by way of example, single ring structures such as cyclopropenyl, cyclobutenyl, cyclopentenyl, cyclooctenyl, cylcooctadienyl and cyclooctatrienyl.

The term “alkynyl” refers to a monoradical of an unsaturated hydrocarbon preferably having from 2 to 22 carbon atoms and having one or more triple bonds (C≡C). Alkynyl groups include ethynyl, propargyl, and the like. Short alkynyl groups are those having 2 to 6 carbon atoms, including all isomers thereof. Long alkynyl groups are those having 8-22 carbon atoms and preferably those having 12-22 carbon atoms as well as those having 12-20 carbon atoms and those having 16-18 carbon atoms.

The term “aryl” refers to a group containing an unsaturated aromatic carbocyclic group of from 6 to 22 carbon atoms having a single ring (e.g., phenyl), one or more rings (e.g., biphenyl) or multiple condensed (fused) rings, wherein at least one ring is aromatic (e.g., naphthyl, dihydrophenanthrenyl, fluorenyl, or anthryl). Aryls include phenyl, naphthyl and the like. Aryl groups may contain portions that are alkyl, alkenyl or akynyl in addition to the unsaturated aromatic ring(s). The term “alkaryl” refers to the aryl groups containing alkyl portions, i.e., -alkylene-aryl and -substituted alkylene-aryly. Such alkaryl groups are exemplified by benzyl, phenethyl and the like.

Alkyl, alkenyl, alkynyl and aryl groups are optionally substituted as described herein and may contain 1-8 non-hydrogen substituents dependent upon the number of carbon atoms in the group and the degree of unsaturation of the group.

The term “alkylene” refers to a diradical of a branched or unbranched saturated hydrocarbon chain, preferably having from 1 to 10 carbon atoms, more preferably having 1-6 carbon atoms, and more preferably having 2-4 carbon atoms. This term is exemplified by groups such as methylene (—CH₂—), ethylene (—CH₂CH₂—), more generally —(CH₂)ₙ—, where n is 1-10 or more preferably 1-6 or n is 2, 3 or 4. Alkylene groups may be branched. Alkylene groups may be optionally substituted. Alkylene groups may have up to two non-hydrogen substituents per carbon atoms. Preferred substituted alkylene groups have 1, 2, 3 or 4 non-hydrogen substituents.

In alkylene groups one or more of the —CH₂— groups may be substituted with an oxygen or sulfur atoms to provide an ether or thioether linker (diradical), such as —(CH₂)ₙ—O—(CH₂)ₘ— or —(CH₂)ₙ—S—(CH₂)ₘ—, where n and m are both integers and n=m is preferably 2-10. Alkylene groups linking nitrogens in the compounds herein may be substituted with one, two, three or more OH or SH groups. Alkylene groups may be branched in that one or more of the —CH₂— groups of the alkylene group may be substituted with one or two alkyl groups (particularly short alkyl groups).

The term “arylene” refers to the diradical derived from aryl (including substituted aryl) as defined above and is exemplified by 1,2-phenylene, 1,3-phenylene, 1,4-phenylene, 1,2-naphthylene and the like.

The term “amino” refers to the group —NH₂ or to the group —NRR where each R is independently selected from the group consisting of hydrogen, alkyl, substituted alkyl, cycloalkyl, substituted cycloalkyl, alkenyl, substituted alkenyl, cycloalkenyl, substituted cycloalkenyl, alkynyl, substituted alkynyl, aryl, heteroaryl and heterocyclic provided that both R's are not hydrogen.

Alkyl groups are optionally substituted as discussed herein and may, dependent upon the size of the alkyl group, have preferably from 1-10 substituent groups. Substituted alkyl groups include those that carry 1 to 8 substituents, 1 to 5 substituents, 1 to 3 substituents, and 1 or 2 substituents.

Haloalkyl” refers to alkyl as defined herein substituted by one or more halo groups as defined herein, which may be the same or different. Representative haloalkyl groups include, by way of example, trifluoromethyl, 3-fluorododecyl, 12,12,12-trifluorododecyl, 2-bromooctyl, 3-bromo-6-chloroheptyl, and the like.

The term “heteroaryl” refers to an aromatic group of from 2 to 22 carbon atoms having 1 to 4 heteroatoms selected from oxygen, nitrogen and sulfur within at least one ring (if there is more than one ring). Heteroaryl groups may be optionally substituted. The term “heterocycle” or “heterocyclic” refers to a monoradical saturated or unsaturated group having a single ring or multiple condensed rings, from 2-22 carbon atoms and from 1 to 6 hetero atoms, preferably 1 to 4 heteroatoms, selected from nitrogen, sulfur, phosphorus, and/or oxygen within at least one ring. Heterocyclic groups may be substituted.

The term “halide” refers to fluoride, chloride, bromide and iodide anions, whereas the term “halo” refers to fluoro, chloro, bromo and iodo groups.

The term “alcohol group” refers herein generally to an organic group that contains one or more OH groups (hydroxide groups) and includes alkyl, alkenyl, alkynyl and aryl groups having one or more OH groups. Organic species carrying two or more OH groups are termed “polyalcohol groups.” An exemplary alcohol group is a —CH₂—OH group.

The term “aminoalcohol” refers herein generally to an organic group that contains one or more OH groups (hydroxide groups) and one or more amino (—NR₂, where R is H or an alkyl, alkenyl or aryl group) groups. The term includes alkyl, alkenyl, alkynyl or aryl groups containing one or more OH groups and one or more —NR₂ groups. Exemplary aminoalcohol groups are alkyl groups having one, two or three OH groups and one —NR₂ group, a specific exemplary aminoalcohol groups is —CH₂—CH₂(OH)—CH₂—NH₂.

The term “ether group” refers to an organic group having at least one C—O—C bond. A “polyether group” refers to an organic group having more than one C—O—C bond. The C—O—C bond may be in a ring or a linear or branched chain. Ether and poly ether groups include among others alkyl, alkenyl or alkynyl groups in which one or more —CH₂— groups are replaced with an O. A specific exemplary ether group is a —CH₂—O—CH₂—CH₂—O—CH₂—CH₃ group.

As to any of the above groups which contain one or more substituents, it is understood, that such groups do not contain any substitution or substitution patterns which are sterically impractical and/or synthetically non-feasible. In addition, the compounds of this invention include all stereochemical isomers arising from the substitution of these compounds.

The compounds of this invention as illustrated in the formulas herein are cationic lipids which contain at least one positive charge, particularly a positively charged N. The nitrogen may be a quaternary nitrogen or a protonated nitrogen. In solution, the cationic lipids of this invention can exist in various charged states dependent upon the number of nitrogens (or other positive charge centers) in the molecule, the number of quaternary nitrogens in the molecule and the protonation state of the molecule. The extent of protonation of the molecule in solution will depend at least in part upon the pH of the medium. Various salts of the compounds of this invention can be prepared. A salt will contain a number of anions (m) of valency (a−) (generally m X^{a-}) sufficient to produce a neutral salt, such that m×a is the number of positive charges in the cationic lipid. Substituent groups may also contain atoms or groups that can be protonated and carry a positive charge. Additional counterions may be needed to form salts of these species. Cationic lipids of this invention in solution may be in the form of cations or polycations.

In general, any anions can be employed in the formation of salts of this invention. Acceptable anions include halides, sulfate, carboxylates, acetates, phosphate, nitrate, trifluoroacetate, glycolate, pyruvate, oxalate, malate, succinicate, fumarate, tartarate, citrate, benzoate, methanesulfonate, ethanesulfonate, p-toluenesulfonate, salicylate and the like. For certain applications pharmaceutically acceptable salts are preferred.

The anion used in the formation of the salts of the cationic lipids described herein may be incorporated into such salts during the synthesis of the cationic lipids described herein, or during a post-synthesis treatment step(s), or combinations thereof.

By way of example only the cationic lipids described herein may be synthesized by:

(1) Treating a polyamine, with an acid chloride of the desired length in the presence of triethylamine and methylene chloride under argon at room temperature to obtain the corresponding substituted amide. By way of example, the polyamine includes, but is not limited to, spermine, and the acid chloride includes, but is not limited to, palmitoyl chloride.

(2) The substituted amide is then reduced in the presence of anhydrous solvent corresponding alkyl amine.

(3) Treatment the alkyl amine with an haloalkyl at high temperature yielded a partially quaternized compound, hich nay be further alkylated using additional haloalkyl to produce the fully quaternized amine compounds with the corresponding halide anion to yield the corresponding cationic lipid salt. By way of example, the haloalkyl includes, but is not limited to, fluoromethane, bromomethane, chloromethane and iodomethane.

Post-synthesis treatment steps used to incorporate an anion into the salt of the cationic lipids described herein, includes, but is not limited to, anion exchange methods. The anion exchange columns used in such methods utilize resins in which the desired anion is available for anion exchange. The type of anion exchange resin used includes, but is not limited to, Dowex® 1×8-200 resin in the chloride form, styrene-divinylbenzene resins with quaternary ammonium exchange sites, polyvinylbenzyltrimethyl ammonium resin, and Dowexe SBR-CL-1. Using such methods the anion of interest may be exchanged for a different anion present, and thereby becoming incorporated into the salt of cationic lipids.

The compounds of this invention may contain one or more chiral centers. Accordingly, this invention is intended to include racemic mixtures, diasteromers, enantiomers and mixture enriched in one or more stereoisomer. The scope of the invention as described and claimed encompasses the racemic forms of the compounds as well as the individual enantiomers and non-racemic mixtures thereof.

Non-cationic lipids, particularly neutral lipids, which are not positively or negatively charged species, are useful in combination with cationic lipids of this invention to form lipid aggregates and more preferably to form liposomes and liposomal compositions. Neutral lipids useful in this invention include, without limitation, Neutral lipids useful in this invention include, among many others: lecithins; phosphotidylethanolamine; phosphatidylethanolamines, such as DOPE (dioleoylphosphatidyl-ethanolamine), POPE (palmitoyloleoyl-phosphatidylethanolamine) and distearoylphosphatidylethanolamine; phosphotidylcholine; phosphatidylcholines, such as DOPC (dioleoylphosphidylcholine), DPPC (dipalmitoylphospha-tidylcholine) POPC (palmitoyloleoyl-phosphatidylcholine) and distearoylphosphatidylcholine; phosphatidylglycerol; phosphatidylglycerols, such as DOPG (dioleoylphospha-tidylglycerol), DPPG (dipalmitoylphosphatidyl-glycerol), and distearoylphosphatidylglycerol; phosphatidyl-serine; phosphatidylserines, such as dioleoyl- or dipalmitoyl-phospatidylserine; diphosphatidylglycerols; fatty acid esters; glycerol esters; sphingolipids; cardolipin; cerebrosides; and ceramides; and mixtures thereof. Neutral lipids also include cholesterol and other 3βOH-sterols.

In another embodiment of the invention, the liposomal composition comprises N,N′,N″,N′″ tetramethyltetrapalmitylspermine (TMTPS) or salts thereof. In a further embodiment of the invention, the liposomal composition comprises N,N′,N″, N′″ tetramethyltetrapalmitylspermine iodide salt. In a still further embodiment of the invention, the liposomal composition comprises N,N′,N″,N′″ tetramethyltetrapalmitylspermine chloride salt.

In another embodiment of the invention, the liposomal composition comprises N,N′,N″,N′″ tetramethyltetrapalmitylspermine or salts thereof and one or more neutral lipids. In a further embodiment of the invention, the liposomal composition comprises N,N′,N″,N′″ tetramethyltetrapalmitylspermine iodide salt and one or more neutral lipids. In a still further embodiment of the invention, the liposomal composition comprises N,N′,N″, N′″ tetramethyltetrapalmitylspermine chloride salt and one or more neutral lipids.

A liposomal composition generally is a formulation that includes one or more liposomes. These formulations are typically colloids, but can be dried formulations as well. A liposome is a vesicular colloidal particle composed of self-assembled amphiphilic molecules. Liposomal compositions of the present invention typically include a cationic lipid and a helper lipid (i.e., a neutral lipid) that are processed using standard methods to form a liposome-containing colloid suspension.

Liposomal compositions of this invention are those containing one or more cationic lipids of this invention, optionally, but preferably in combination with one or more neutral and/or helper lipids which are formed by standard methods known in the art to form liposomes. The liposomal compositions are formed, for example, by sonication, extrusion, reverse evaporation, microfluidization and like methods. Liposomal compositions can be distinguished one from another by particle size measurements. Different compositions will exhibit differences in particle size and uniformity of particle size, e.g., average particle size, and polydispersity. Different compositions will exhibit differences in the extent of the composition that is in the form of liposomes. Preferred liposomal compositions will exhibit particle size in the range 120 nm and 800 nm and will exhibit generally lower polydispersity.

Preferred liposomal compositions of the present invention comprise uniformly-sized liposome particles. By uniformly-sized, it is meant that a large number of the lipids are in the form of liposomes. In one embodiment, at least 75% of the liposomal composition, more preferably 85%, even more preferably 90% or above, is in the form of liposome particles ranging between 120 nm and 800 nm. The uniformity and size of the liposomal composition can be determined using dynamic laser light scattering or visually through an electron microscope.

Cellular Delivery (or delivery) refers to a process by which a desired compound is transferred to a target cell such that the desired compound is ultimately located inside the target cell, or in or on the target cell membrane. In certain applications delivery to a specific target cell is preferable. In many uses of the compounds of the invention, the desired compound is not readily taken up by the target cell or appropriate cytoplasmic compartment and delivery via liposomal compositions is a means for getting the desired compound into the cell cytoplasm.

Methods of this invention relate to the delivery of one or more molecules to a target cell. The molecules may be macromolecules which generally include peptides, polypeptides, nucleic acids and various biologically active molecules or compositions. Preferably the biologically active molecule delivered retains biological activity on delivery.

A target cell is most generally any cell into which a desired chemical species (compound, complex, composition, or molecule) is to be delivered. Cells to which the delivery methods of this invention can be applied include cells in vitro, cells ex vivo or cells in vivo. Target cells may be cell culture, on tissue culture, in any form of immobilized state, or grown on liquid, semi-solid or solid medium. Target cells may be in the form of a monolayer. Target cells may be collected from an organism and/or cultures by any known method. Target cells include cells without cell walls and cells from which cell walls have been removed by any known treatment (e.g., formation of protoplasts) from which viable cells can be recovered. Cells include those that are adapted for growth as non-adherent, suspension cell cultures. Cells include any and all eukaryotic cells, including among others, animal cells, mammalian cells, including human cells. Cells include cells from primary cell cultures and cells of established cell lines. Target cells include, among others, suspension CHO cells, A549 cells, NIH3T3 cells, HeLa cells, including suspension HeLa cells, HEK293-MSR cells, including suspension HEK293 cells, PC12 cells, HepG2 cells, Jurkat cells, U937 cells, COS-7 cells, Vero cells, BHK cells and ME-180 cells. The methods and materials of this invention are particularly useful for transfection of suspension CHO cells.

Cells useful in the present invention may be provided as freshly prepared cells derived from a subject or biological source or as cultured cells, and in certain preferred embodiments the cells are cultured cells. As known in the art, cultured cells may be adherent cells that naturally adhere to a solid substrate, or may be non-adherent cells that may be maintained as cells in a suspension of freely growing cells by cultivation in an appropriate cell culture system. Liposomal transfection agents of this invention are particularly useful for transfection of non-adherent cells, most particularly CHO cells in suspension culture.

Biologically active (bioactive) refers to a composition, complex, compound or molecule which has a biological effect or that it modifies, causes, promotes, enhances, blocks or reduces a biological effect, or which enhances or limits the production or activity of, reacts with and/or binds to a second molecules which has a biological effect. The second molecule can, but need not be, an endogenous molecule (e.g., a molecule, such as a protein or nucleic acid, normally present in the target cell). A biological effect may be, but is not limited to, one that stimulates or causes an immunoreactive response; one that impacts a biological process in a cell, tissue or organism (e.g., in an animal); one that imparts a biological process in a pathogen or parasite; one that generated or causes to be generated a detectable signal; one that regulates the expression of a protein or polypeptide; one that stops or inhibits the regulation of expression of a protein or polypeptide; or one that causes or enhances the expression of a protein or polypeptide. Biologically active compositions, complexes, compounds or molecules may be used in investigative, therapeutic, prophylactic and diagnostic methods and compositions and generally act to cause.

The materials and methods of this invention can be employed in cell-based assays to facilitate identification, quantitation and/or assessment of biologically-active compositions, complexes, compounds or molecules. For example, cell-based assays can employ cells in which a biological effect or response can be measured or observed on exposure of the cell or on introduction into the cell of an appropriate biologically active compound, complex, composition or molecule. The methods and materials of this invention can, for example, be employed to prepare cells useful in such cell-based assays by introduction of an appropriate reporter gene expression construct to produce a transiently expressing reporter cell. Alternatively or in combination, the methods and materials of this invention can be employed, for example, to introduce one or more chemical species (e.g., compounds, complexes, compositions or molecules) into a cell, e.g., nucleic acids, siRNA, etc., where a biological effect caused by that species generates any measurable response or signal and allows identification of species exhibiting such biological effect or allows assessment or comparison of the extent or magnitude of the biological effect caused by different chemical species.

Cell-based assays are any assays that are based on the use of live cells wherein a change in cells is measured or detected as the basis of the assay. Cell-based assays are useful for detection or measurement of activity of any one or more chemical species (compound, composition, complex, or molecule) within the cell employed in the assay. Activity is assessed by measurement or detection of changes in cell proliferation, cell viability, cell death, cell toxicity, cell motility, cell morphology, or of the production of a product by the cell. For example, activity may be assessed by detection of expression of a reporter gene from a reporter gene expression construct in a cell. Methods and materials of this invention are useful in the preparation of cells for use in cell-based assays, for example, for transfection of a cell with an appropriate reporter gene expression construct to produce a cell which can express the reporter. Methods and materials of this invention can also be employed in conducting cell-based assays for delivery of chemical species, e.g., nucleic acids, to a cell in such an assay for assessment of their activity in the cell. Cell-based assays can be used to assess biological activity of one or more members of a library of chemical species in a living cell. For example, the effect of the chemical species on cell proliferation, cell viability, cell death, cell toxicity, cell motility, cell morphology, levels of expression of a reporter gene, levels of expression of a detectible gene product, and/or levels of production of any measurable cell product can be assessed in a cell-based assay. Cell-based assays can be conducted in cells in suspension, or cells grown on a plate. Cell-based assays can be adapted and automated as needed, as is known in the art, for high-through-put screening.

Transfection is the delivery of expressible nucleic acid to a target cell, such that the target cell is rendered capable of expressing the nucleic acid. The term expression means any manifestation of the functional presence of the nucleic acid within the cell, including without limitation both transient expression and stable expression. Expression is the process by which a gene produces a polypeptide. It includes transcription of the gene into messenger RNA (mRNA) and the translation of such mRNA into polypeptide.

The efficiency of transfection is assessed in the art in several ways. For example, transfection efficiency can be assessed by measurement of the amount of expression of a transfected gene in a cell population. Efficiency of transfection can be assessed by determining the percent of cells in a transfected cell population that express a detectible level of expression of the transfected gene. The latter method of assessing transfection efficiency is the method preferred for use herein. Preferred transfection agents exhibit detectible levels of transfected gene expression in 10% or more of the cells in a transfected cell population. More preferred transfection agents exhibit detectable levels of transfected gene expression in 25% or more of the cells in a transfected cell population. Even more preferred transfection agents exhibit detectible levels of transfected gene expression in 50% or more of the cells in a transfected cell population.

A transfection reagent is any substance which provides significant enhancement of transfection (2-fold or more) over transfection compositions that do not comprise the transfection reagent. Cationic lipids of this invention, mixtures of cationic lipids of this invention and mixtures of one or more cationic lipids of this invention in combination with one or more neutral lipids and/or one or more helper lipids of this invention are exemplary transfection reagents. Transfection reagents of this invention are preferably in the form of liposomal compositions.

The term nucleic acid includes both DNA and RNA without regard to molecular weight or source. Nucleic acids include the full range of polymers of single or double stranded nucleotides. A nucleic acid typically refers to a polynucleotide molecule comprised of a linear strand of two or more nucleotides (deoxyribonucleotides and/or ribonulceotides) or variants, derivatives and/or analogs thereof. The exact size of nucleic acid employed will depend upon the application and many other factors, as is well known in the art. Nucleic acids include without limitation primers, probes, oligonucleotides, antisense oligonulceotides, constructs, plasmids, vectors, genes or gene fragments, transgenes, genomic DNA, c-DNA, PCR products, restriction fragments and the like. Nucleic acids may be derived from any natural source or may be synthetic. A DNA molecule is any DNA molecule of any size, from any source, including DNA from viral, prokaryotic and eukaryotic organisms, as well as synthetic DNA. DNA molecules include without limitation single-stranded probes, expression constructs, expression cassettes, reported gene constructs, plasmids and vectors. A DNA molecule is any DNA molecule of any size, from any source, including DNA from viral, prokaryotic and eukaryotic organisms, as well as synthetic DNA and variants, derivatives and analogs thereof. A RNA molecule is any RNA molecule of any size, from any source, including RNA from viral, prokaryotic and eukaryotic organisms, as well as synthetic RNA and variants, derivatives and analogs thereof. RNA molecules include without limitation m-RNA, t-RNA, ribozymes, aptamers, micro RNA, plasmid-based RNAi, short RNA duplexes, chemically-modified synthetic RNA. Nucleic acid derivatives include without limitation labeled nucleic acids, nucleic acids that are conjugated to tracking dyes. The methods and materials of this invention can be employed for the delivery of any such nucleic acids to cells. The methods and materials of this invention can be employed to transfect cells with expressible nucleic acid such that there is a detectible level of expression of that nucleic acid in the cell.

A vector is a nucleic acid molecule that provides a useful biological or biochemical property to a nucleic acid sequence or molecule of interest, for example, an Insert, a coding region, etc. Examples include plasmids, phages, autonomously replicating sequences (ARS), centromeres, and other nucleic acid sequences that are able to replicate or be replicated in vitro or in a host cell, or to convey a desired nucleic acid segment to a desired location within a host cell. A vector may comprise various structural and/or functional sequences, for example, one or more restriction endonuclease recognition sites at which the vector sequences can be manipulated in a determinable fashion without loss of an essential biological function of the vector, and into which a nucleic acid fragment can be inserted, for example to bring about its replication and/or cloning. Vectors can further provide primer sites, e.g., for PCR, transcriptional and/or translational initiation and/or regulation sites, recombinational signals, replicons, selectable markers, and other sequences known to those skilled in the art. A vector comprising a nucleic acid insert is a construct. Thus, a gene therapy construct is a gene therapy vector into which a therapeutic gene has been cloned. Similarly, a construct that expresses an antisense transcript is an “antisense construct.”

A cloning vector is a plasmid, cosmid, viral, or phage DNA or other DNA molecule which is able to replicate autonomously in a host cell, into which DNA may be spliced without loss of an essential biological function of the vector, in order to bring about its replication and cloning. The cloning vector may further contain a marker suitable for use in the identification of cells transformed with the cloning vector. Markers may be, for example, antibiotic resistance genes, e.g., tetracycline resistance or ampicillin resistance. Various methods known in the art for inserting a desired nucleic acid fragment can be applied to clone a fragment into a cloning vector to be used in the present invention. A cloning vector can further contain one or more selectable markers suitable for use in the identification of cells transformed with the cloning vector. A cloning vector comprising a circular or linear nucleic acid molecule which includes preferably an appropriate replicon is a subcloning vector. A subcloning vector can also contain functional and/or regulatory elements that are desired to be incorporated into the final product to act upon or with the cloned DNA Insert. Additionally or alternatively, the subcloning vector can also contain a Selectable marker (preferably DNA). An expression vector is a vector similar to a cloning vector but which is capable of enhancing the expression of a gene which has been cloned into it, after transformation into a host. The cloned gene is usually placed under the control of (i.e., operably linked to) certain transcriptional regulatory sequences such as promoter sequences. An expression vector comprising an operably linked nucleic acid insert is an “expression construct.”

Vectors can contain genes or portion thereof, usually included to provide a necessary function to the maintenance of the vector (e.g., genes required for DNA replication) or otherwise included on the vector in order to identify, distinguish or select cells comprising the vector or desired constructs prepared from the vector. Non-limiting examples of such genes are selectable markers.

A reporter gene is a nucleic acid encoding a readily assayable protein. Assays can be qualitative, quantitative, manual, automated, semi-automated, etc. Non-limiting examples of reporter genes include: genes encoding α-galactosidase (lacZ), neomycin resistance, HIS3, luciferase (LUC), chloramphenicol acetyltransferase (CAT), β-glucuronidase (GUS), human growth hormone (hGH), alkaline phosphatase (AP), secreted alkaline phosphatase (SEAP), and fluorescent polypeptides such as GFP. Those skilled in the art will be able to select reporter genes appropriate for the host cell and application of interest. For reviews of vectors and reporter genes see Baneyx F. Recombinant protein expression in Escherichia coli. Curr Opin Biotechnol 10:411-421, 1999; Van Craenenbroeck K, Vanhoenacker P, Haegeman G. Episomal vectors for gene expression in mammalian cells. Eur. J. Biochem. 2000 September;267(18):5665-78; Soll D R, Srikantha T. Reporters for the analysis of gene regulation in fungi pathogenic to man. Curr Opin Microbiol. 1998 August; 1 (4):400-5; Possee R D. Baculoviruses as expression vectors. Curr Opin Biotechnol. 1997 October;8(5):569-72; and Mount R C, Jordan B E, Hadfield C. Reporter gene systems for assaying gene expression in yeast. Methods Mol. Biol. 1996;53:239-48.

The term kit refers to transfection or protein expression kits which include one or more of the compounds of the present invention or mixtures thereof. Such kits may comprise a carrying means being compartmentalized to receive in close confinement one or more container means such as vials, test tubes and the like. Each of such container means comprises components or a mixture of components needed to perform transfection. Such kits may include one or more components selected from nucleic acids (preferably one or more vectors), cells, one or more compounds of the present invention, lipid-aggregate forming compounds, transfection enhancers, biologically active substances, etc.

The invention also provides kits comprising the one or more liposomal compositions of this invention. Such kits typically comprise a carrier, such as a box, carton, tube or the like, having in close confinement therein one or more containers, such as vials, tubes, ampules, bottles and the like, wherein a first container contains one or more of the liposomal compositions of the present invention. The kits encompassed by this aspect of the present invention may further comprise one or more additional components (e.g., reagents and compounds) necessary for carrying out one or more particular applications of the compositions of the present invention. For example the kit may contain one or more components useful for the carrying out a cell-based assay. In further examples, the kits may contain one or more components useful in carrying out a desired transfection of non-adherent, suspension cells. For example, the kit can include a vector, such as an expression vector. In yet further examples, the kit may contain one or more components useful in carrying out diagnosis, treatment or prevention of a particular disease or physical disorder (e.g., one or more additional therapeutic compounds or compositions, one or more diagnostic reagents). In general kits may also contain one or more buffers, control samples, carriers or recipients, and the like, one or more additional compositions of the invention, one or more sets of instructions, and the like.

This invention also includes transfection kits which include one or more of the compounds or compositions of the present invention or mixtures thereof. Particularly, the invention provides a kit comprising one or more of the compounds of the present invention and at least one additional component selected from the group consisting of a cell, cells, a cell culture media, a nucleic acid, and instructions for transecting a cell or cells.

One embodiment of the present invention provides a method for introducing a macromolecule into one or more cells comprising the steps:
(a) forming a liposomal composition comprising one or more cationic lipids, or mixtures thereof, where the cationic lipids have the formula: 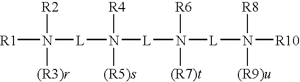
   or salts and polycations thereof, where r, s, t and u independent of one other are 0 or 1 to indicate the presence or absence of the individual group, wherein when N is tetravalent it is positively charged, and wherein at least one of r, s, t or u is 1;
   where L is a divalent organic radical independently selected from the group consisting of an alkylene group having from 1 to about 8 carbon atoms, wherein one or more non-neighboring —CH₂— groups can be replaced with an O or S atom, and wherein one or more carbon atoms of the group can be substituted with an OH, SH, SR or OR group, where R is an alkylene group having from 1 to about 6 carbon atoms;
   where R1-R10 are independently selected from the group consisting of hydrogen and a C₁₋₂₂ alkyl, C₂₋₂₂ alkenyl, C₂₋₂₂ alkynyl and C₁₋₂₂ aryl, optionally substituted with one or more of an alcohol, aminoalcohol, hydroxyl, amine, carbohydrate, ether, polyether, amide, polyamide, ester, mercaptan, urea, thiourea, heterocyclic group, or heterocyclic aromatic group;
where between 2 and 4 groups of R1-R10 are selected from the group consisting of a C₈₋₂₂ alkyl, C₈₋₂₂ alkenyl, C₈₋₂₂ alkynyl and C₈₋₂₂ aryl, optionally substituted with one or more of an alcohol, aminoalcohol, hydroxyl, amine, carbohydrate, ether, polyether, amide, polyamide, ester, mercaptan, urea, thiourea, heterocyclic group, or heterocyclic aromatic group;
where between 0 and 6 groups of R1-R10 are independently selected from the group consisting of a C₁₋₆ alkyl, C₂₋₆ alkenyl and C₂₋₆ alkynyl;
where not all of R1, R2, R3, R8, R9 and R10 are hydrogens; and
   one or more neutral lipids, wherein the molar ratio of said one or more cationic lipids to said one or more neutral lipids in said liposomal composition is between 1:0.8 and 1:3.0;
(b) combining said liposomes with said macromolecule to form a liposome-macromolecule complex;
(c) contacting one or more cells with the liposome-macromolecule complex to thereby introduce the macromolecule into the one or more cells.

Preferably, the macromolecule is a negatively charged molecule. For example, the macromolecule is preferably a nucleic acid such as RNA or DNA, or a protein or polypeptide.

Preferably, R1 and R10 independently are selected from the group consisting of an alkyl having from about 1 to about 22 carbon atoms; where R2, R3, R5, R7, R8 and R9 independently are selected from the group consisting of hydrogen and an alkyl having from 1 to about 6 carbon atoms; and where R4 and R6 independently are selected from the group consisting of an alkyl, alkenyl and alkynyl having from 2 to about 22 carbon atoms wherein one or more non-neighboring —CH₂— groups can be replaced with an O or S atom. More preferably, R1, R3, R9 and R10 are selected from the group consisting of hydrogen and an alkyl having 1 to about 3 carbon atoms; R2, R4, R6 and R8 are selected from the group consisting of an alkyl having about 12 to about 20 carbon atoms, and R5 and R7 are CH₃. More preferably, L is selected from the group consisting of an alkyl having 2 to about 4 carbon atoms; R1, R5, R7 and R10 are CH₃; R3 and R9 are hydrogen; and R2, R4, R6 and R8 are an alkyl having 16 carbon atoms.

One embodiment of the present invention provides a method for introducing a macromolecule into one or more cells comprising the steps:
(a) forming a liposomal composition comprising one or more cationic lipids, or mixtures thereof, where the cationic lipids have the formula: 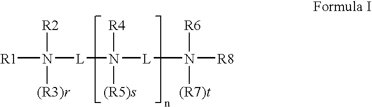
or salts or polycations thereof, where wherein when N is tetravalent it is positively charged, and wherein at least one of r or t is 1 or at least one s is 1 (the lipid carries at least one positive charge);
   where L is a divalent organic radical independently selected from the group consisting of a C₁₋₁₀ alkylene group, wherein one or more non-neighboring —CH₂— groups can be replaced with an O or S atom, and wherein one or more carbons of the alkyl group can be substituted with an OH, SH, SR or OR group where R is an alkyl group having from 1 to about 6 carbon atoms;
   where n is an integer from 0 to 10;
   where R1-R8 are independently selected from the group consisting of hydrogen and a C₁₋₂₂ alkyl, C₂₋₂₂ alkenyl, C₂₋₂₂ alkynyl and C₆₋₂₂ aryl, optionally substituted with one or more of an alcohol, aminoalcohol, hydroxyl, amine, carbohydrate, ether, polyether, amide, polyamide, ester, mercaptan, urea, thiourea, heterocyclic group, or heterocyclic aromatic group;
   where at least two of R1-R8 are independently selected from the group consisting of a C₈₋₂₂ alkyl, C₈₋₂₂ alkenyl, C₈₋₂₂ alkynyl and C₈₋₂₂ aryl, optionally substituted with one or more of an alcohol, aminoalcohol, hydroxyl, amine, carbohydrate, ether, polyether, amide, polyamide, ester, mercaptan, urea, thiourea, heterocyclic group, or, heterocyclic aromatic group; and
   where at least two of R1-R8 are independently selected from the group consisting of a C₁₋₆ alkyl, C₂₋₆ alkenyl and C₂₋₆ alkynyl;
where when n is 0, none of R1-R8 is an alkyl group substituted with an aminoalcohol and when n is 1 not all of R1-R3 and R6-R8 can be hydrogen; and
one or more neutral lipids, wherein the molar ratio of said one or more cationic lipids to said one or more neutral lipids in said liposomal composition is between 1:0.8 and 1:3.0;
(b) combining said liposomes with said macromolecule to form a liposome-macromolecule complex;
(c) contacting one or more cells with the liposome-macromolecule complex to thereby introduce the macromolecule into the one or more cells.

Preferably, the macromolecule is a negatively charged molecule. For example, the macromolecule is preferably a nucleic acid such as RNA or DNA, or a protein or polypeptide.

Preferably, R1 and R8 are independently selected from the group consisting of a hydrogen and a C₁₋₈ alkyl, C₂₋₈ alkenyl, C₂₋₈ alkynyl and C₂₋₈ aryl, optionally substituted by one or more of an alcohol, aminoalcohol, hydroxyl, amine, carbohydrate, ether, polyether, amide, polyamide, ester, mercaptan, urea, thiourea, heterocyclic group, or heterocyclic aromatic group; R2, R4, and R6 are independently selected from the group consisting of hydrogen and a C₁₋₂₂ alkyl, C₂₋₂₂ alkenyl, C₂₋₂₂ alkynyl and C₁₋₂₂ aryl, optionally substituted with one or more of an alcohol, amine, amide, ether, polyether, polyamide, ester, mercaptan, urea, or thiol; and R3, R5 and R7 are independently selected from the group consisting of hydrogen and a C₁₋₆ alkyl.

In one embodiment of the present invention, the one or more neutral lipids comprises cholesterol or DOPE or a mixture thereof. In further embodiment, the molar ratio of the one or more cationic lipids to the one or more neutral lipids in the transfection reagents is between 1:1.6 and 1:2.3. In further embodiment, the molar ratio of the one or more cationic lipids to the one or more neutral lipids in the transfection reagents is between 1:1.6 and 1:1.9.

In another embodiment of the present invention, the molar ratio of the one or more cationic lipids to the one or more neutral lipids in the transfection reagents is between about 1:1.5 and 1:1.7. In another embodiment, the molar ratio of the one or more cationic lipids to the one or more neutral lipids in the transfection reagents is 1:1.5.

In one embodiment, the cells are contained in a non adherent suspension cell culture. Preferably, the liposome-macromolecule complex has a concentration between about 0.1 μg of lipid/ml of said suspension cell culture and about 10 μg of lipid/ml of said suspension cell culture when added to the cells. More preferably, the liposome-macromolecule complex has a concentration between about 1.0 μg/ml and about 5.0 μg/ml. More preferably, the liposome-macromolecule complex has a concentration of about 3.0 μg/ml. If the macromolecule is a nucleic acid, the final concentration of the nucleic acid is preferably 0.1 μg/ml to 10 μg/ml, more preferably 1.0 μg/ml.

The temperature employed in methods herein can be any temperature at which the viability of the cells can be maintained and can commonly range from just below room temperature (20° C.) to above room temperature (40° C.). Transfection method steps can be performed at about room temperature. The temperature can also vary during the transfection process. For example, preparing the liposome-macromolecule complex can be conducted at room temperature, approximately 25° C., and then the target cells can be incubated for 24 to 72 hours at 37° C. after the liposome-macromolecule complex has been added to the cells.

Once the transfection reagent is diluted in the cell media, the transfection reagent is incubated for 25 minutes or less before mixing with the macromolecule. Preferably, the transfection reagent is incubated for 15 minutes or less. More preferably, the transfection reagent is incubated for 5 minutes or less. Once the macromolecule is diluted in the cell media, the macromolecule is incubated for a period of time which does not significantly degrade the macromolecule. For example, nucleic acids can be incubated for 5 to 30 minutes after dilution before being mixed with the diluted transfection reagent. Once the transfection reagent and macromolecule have been mixed together, the transfection reagent-macromolecule complex is incubated for 5 to 30 minutes, preferably for 15 to 25 minutes, before adding to the target cells.

After treatment with the transfection reagent and macromolecule, the target cells are incubated for a selected amount of time sufficient to exhibit a biological effect of transfection, e.g., to exhibit expression of a polypeptide or protein. For example, transfected cells are incubated for a sufficient time to generate a desired amount of a protein or polypeptide expressed from nucleic acid introduced into the cells. This time can vary significant dependent upon the specific cell, nucleic acid or protein that is involved. Typically incubation can vary from hours to days. More specifically incubation can vary from overnight (e.g., 12-18 hours), 24 hours to multiples of days. Preferably, cells are incubated for 24 to 72 hours after treatment with the liposome-macromolecule complex.

The cell density will depend on the type of cell and transfection reagent to be used. In general, cell density can vary from 1.0×10⁴ to 1.5×10⁶ cells/ml of cell media. The cell media can be any call media known in the art able to maintain viability of the specific cells. If a well plate is used, the number of wells and volume, i.e., a 96-well plate versus a 48-well plate, can also affect the optimum cell density and reagent concentration.

Using the compositions and methods of the present invention, compounds that may be suitable as pharmaceutical candidates can be rapidly, efficiently, and sensitively screened for their abilities to affect a cellular response, thereby identifying compounds that may have significant therapeutic, prognostic and preventative uses in treating and preventing a variety of diseases and physical disorders. Compounds that can be identified as pharmaceutical candidates according to the methods of the present invention encompass numerous inorganic or organic chemical types and classes, although typically they are organic molecules. Non-limiting examples of such compounds include small organic compounds having a molecular weight of more than 100 and less than about 10,000 daltons, preferably less than about 2000 to 5000 daltons, and may comprise one or more functional groups such as one or more amine, imine, amide, hydrazine, sulfhydryl, alkyl, alkenyl, alkanoyl, carbonyls, hydroxyl or carboxyl groups, and preferably at least two of the functional chemical groups, as well as cyclical carbon or heterocyclic structures, and/or aromatic or polyaromatic structures substituted with one or more of the above functional groups. Candidate compounds or pharmaceutical agents that are identifiable using the compositions and methods of the present invention can be obtained from a wide variety of sources, including libraries of synthetic or natural compounds. Such libraries contain test compounds, the biological activity of which is to be assessed in cell based assays.

The following examples are intended to illustrate but not limit the invention.

### Synthesis of TMTPS-Iodide

### EXAMPLE 1

In one embodiment of the invention, the transfection reagent comprises N, N′,N″, N′″ tetramethyltetrapalmitylspermine (TMTPS-Iodide). To prepare TMTPS-Iodide, 3.88 g of spermine (Sigma Cat. #S 3256) is first added to a 3 liter round bottom flask. 2000 ml of chloroform is added to the flask, followed by 8.6 ml of triethylamine (Aldrich Cat. #13,206-3). A rubber septum is attached to the flask opening and an argon balloon attached to the septum. The flask is set in an ice bath and cooled for 30 minutes. Palmitoyl chloride, 19.2 ml (4.2 molar equivalent) Aldrich Cat. #P7-8, was slowly added to the flask with a syringe. As an alternative, an increased amount of palmitoyl chloride 27.7 ml (4.8 molar equivalent) can be added.

The flask is removed from the ice bath, and the reaction allowed to proceed for 48-72 hours at room temperature with stirring. The reaction is analyzed using THF(1):CH₂Cl₂(2). The flask is set in an ice bath and cooled for approximately 30 minutes. The argon balloon is removed and chloroform is added to bring the total volume to 2.5 L. A minimum of 200 ml of 10% sodium bicarbonate in water is added and the contents of the flask stirred for 15 minutes. The contents of the flask are transferred to a 2 liter separatory funnel where the aqueous phase is removed from the organic phase. The organic phase is washed two more times with a minimum of 200 ml of 10% sodium bicarbonate. The organic phase is washed twice with 200 ml of 1 M HCl. The organic phase is transferred to a 2 liter Erlenmeyer flask and dried by adding 20 g of sodium sulfate, mixing gently and allowing to sit approximately 15 minutes. The organic phase can be spotted on a TLC plate and developed with THF(1):CH₂Cl₂(2). When visualized with 3% H₂SO₄ in MeOH, one spot is observed at Rf 0.9. The organic phase is filtered and transferred to a round bottom flask and evaporated on a rotary evaporator to produce approximately 20 g of a white solid (N, N′,N″, N′″ tetrapalmitoylspermine).

The tetrapalmitoylspermine is dissolved with 2.5 L of anhydrous tetrahydrofuran (THF) in a 3 liter round bottom flask. Under a blanket or argon, 15 g of lithium aluminum hydride (Aldrich Cat. #19,987-7) is slowly added to the flask. The suspension is refluxed under argon for 34 days. The flask is set in an ice bath and cooled for approximately 30 minutes. Sodium hydroxide (100 ml of 0.5 M solution) is slowly added to the flask. The contents of the flask are stirred for approximately 24 hours until a white solid sticks to the walls of the flask. Using a coarse filter funnel, the liquid is decanted into a 3 liter Erlenmeyer flask. The white solid is rinsed three times with 100 ml of THF and the rinses collected with the decanted liquid. The THF solution is dried used 50 g of sodium sulfate and filtered. The solution is rotary evaporated resulting in approximately 18 g of a waxy, white solid (N,N′,N″,N′″ tetrapalmytylspermine).

The tetrapalmytylspermine is dissolved in 200 ml of iodomethane (Aldrich Cat. #28,956-6) in a 3 liter round bottom flask and stirred for 24-72 hours at room temperature. Iodomethane is removed by rotary evaporation and the residue is redissolved in 1 L of CH₂Cl₂. The CH₂Cl₂ solution is washed twice with 200 ml of 10% saturated sodium bicarbonate solution in water. The organic phase is dried with 50 g of sodium sulfate and filtered. The solution is rotary evaporated resulting in approximately 22.5 g of TMTPS-Iodide.

This process produces a mixture of lipid compounds in addition to TMTPS-Iodide. This mixture is used without further separation or purification of the components. The lipid compounds of the mixture have a similar structure to TMTPS-Iodide but may have no methyl groups, two or more methyl groups, or up to six methyl groups. The compounds will have a maximum of four long fatty acids. TMTPS-Iodide is the major component, representing at least 50% and possible 70% or more of the mixture.

### Formation of Cationic Lipid-Neutral Lipid Protocol

### EXAMPLE 2

1.1163 g TMTPS-Iodide (from example 1) and 0.8846 g of DOPE are added to a two liter round bottom flask. Approximately 100 ml of methylene chloride is added and the contents of the flask are swirled or shaken until all of the lipid dissolves. The methylene chloride is evaporated on a rotary evaporator for approximately 10 minutes and the flask is attached to a high vacuum pump overnight to form a lipid film on the inner surface of the flask.

### Formation of Liposome Protocol

### EXAMPLE 3

1,000 ml of distilled water is added to the flask having the lipid film from example 2. This will achieve a concentration of 2.0 mg/ml. The contents of the flask are swirled or shaken until all of the lipid is dislodged from the surface of the flask. The lipid suspension is passed through a microfluidizer (Models 110Y and 110T, Microfluidics Corp., Newton, Mass.) at a flow rate of 330±10 ml/min at approximately 60 psi. The lipid suspension is collected in an autoclaved 2 liter Erlenmeyer flask and passed through the microfluidizer four more times (for a total of 5 passes). After the final pass, the lipid suspension is collected in an autoclaved 2 liter Erlenmeyer flask.

The concentration of the liposome formulation is checked by thermogravimetric analysis (using a Perkin Elmer, model TGA7 instrument) and the concentration is adjusted to 2.0 mg/ml with distilled water. The particle size of the liposome formulation is checked using a particle analyzer (dynamic light scattering device, Model 90 Plus, Brookhaven Instruments Corp., Novato, Calif.). The width of the particle sizes should range from 60-1500 nm in diameter, with an average diameter between 200-700 nm. The liposome formulation is stored at 4° C.

### CHO—S Suspension Cell Transient Transfection Protocol

### EXAMPLE 4

CHO—S cells (Gibco Catalogue #10743-029) are cultivated in a humidified 37° C., 8% CO₂ environment using an orbital shaker (New Brunswick Scientific INNOVA Model #2000-2300) at 125-145 rpm. The CD CHO media (Gibco Cat. #10743-011 or 10743-029) contains 10 ml/L sodium hypoxanthine and thymidine (HT) supplement (Gibco Cat. #11067-030), and 40 ml/L L-glutamine (200 mM) (Gibco Cat. #25030-081). Cell densities are maintained from 0.02-1×10⁶ cells/ml in a shaking or bioproduction tissue culture suspension system prior to transfections. 3 L Corning Erlenmeyer flasks are used for 1 L cultures.

The transfection reagent, 8 to 20 μl, (stock concentration of 0.5 to 2 mg of lipid/ml) is diluted into 80 μl of OptiPro™ SFM (Gibco Cat. #12309-019) for every 3 ml of final CHO—S culture volume to be transfected (final concentration of the lipid is approximately 1 μg lipid to 1 ml suspension culture). The transfection reagent dilution is incubated for no longer than 5 minutes at room temperature. One microgram of purified DNA is diluted in 100 μl of OptiPro™ SFM for every 1 ml of final CHO—S culture volume to be transfected. The DNA dilution is incubated until the transfection reagent dilution is ready. The DNA dilution and transfection reagent dilution are mixed together by inversion and incubated for 15 to 25 minutes at room temperature. The CHO—S cell culture is diluted in growth medium to 0.3×10⁶ cells/ml and placed in appropriate vessels for orbital shakers or other apparatus for suspension cultures. The transfection reagent-DNA dilution mixture is added directly into the CHO—S cell culture to a final concentration of nucleic acid of 0.1 to 10 μg/ml suspension culture, typically about 1 μg/ml suspension culture, and incubated at 37° C., 8% CO₂ until harvesting at 24-72 hours post transfection.

This protocol can be modified for the plating of CHO—S transfected cell cultures. After transfection with the transfection reagent-DNA dilution mixture, the CHO—S suspension culture is incubated for about 2 hours at 37° C., 8% CO₂. The cell culture is counted and the cell viability is determined. The culture is centrifuged at 1-1.8 k rpm for about 10 minutes. The supernatant is aspirated and washed with 1×PBS (without CaCl₂ and MgCl₂ Gibco Cat. #14190-144). The amount of PBS used should be at least half of the original media volume. The culture is centrifuged again at 1-1.8 k rpm for about 10 minutes and re-suspended in complete DMEM with 10-20% FBS to the cell density desired for plating. For 96-well plates, the cell density used is 1.6-3×10⁴ cells/100 μl/well. The cell line will adapt well to adherent culture within 24-48 hours.

### Comparative Results

### EXAMPLE 5

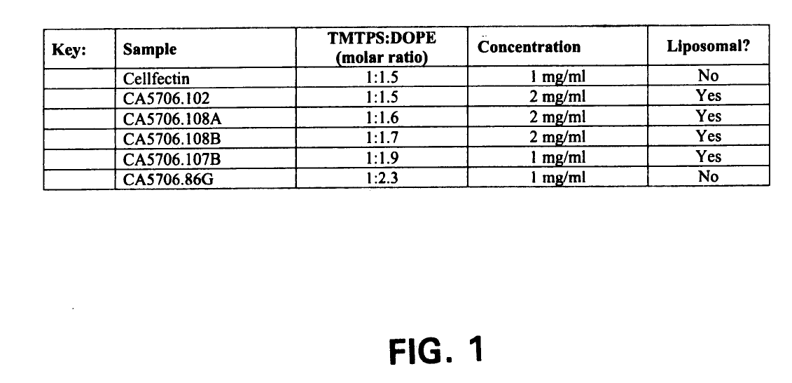

The transfection efficiencies of TMTPS-Iodide:DOPE liposomal transfection reagents of the present invention were compared to Cellfectin™, a non-liposomal TMTPS-Iodide:DOPE reagent. See FIG. 1.

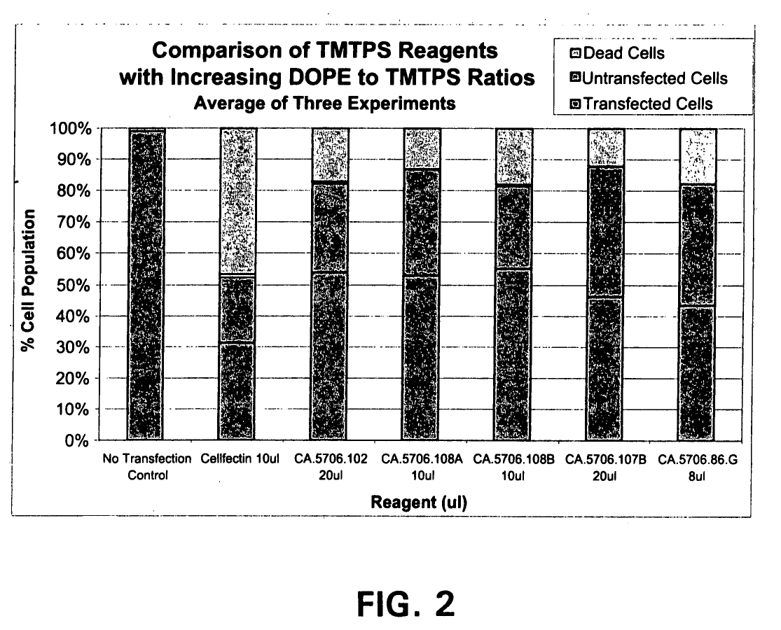

FIG. 1 is based on a three experiment average using the reagents to transfect suspension CHO cells according to the protocol described in Example 4. The concentration of 1 mg/ml or 2 mg/ml in FIG. 1 represents the initial starting concentration of the reagent that produced the optimal transfection efficiency. FIG. 2 shows the resulting toxicity and transfection efficiency of the different transfection reagents and concentrations.

Cellfectin™ has a molar ratio of TMTPS-Iodide to DOPE of 1:1.5. When used to transfect CHO suspension cells, approximately 30% of the cells are transfected and there is approximately 45% cell death. A non-liposomal composition of TMTPS-Iodide:DOPE having a molar ratio of 1:2.3 results in approximately 18% cell death with approximately 43% of the cells being transfected. A liposomal reagent of the present invention where the molar ratio of TMTPS-Iodide:DOPE is 1:1.5 results in approximately 18% cell death with approximately 53% of the cells being transfected. A liposomal reagent of the present invention where the molar ratio of TMTPS-Iodide:DOPE is 1:1.6 results in approximately 13% cell death with approximately 52% of the cells being transfected. A liposomal reagent of the present invention where the molar ratio of TMTPS-Iodide:DOPE is 1:1.7 results in approximately 19% cell death with approximately 55% of the cells being transfected. A liposomal reagent of the present invention where the molar ratio of TMTPS-Iodide:DOPE is 1:1.9 results in approximately 12% cell death with approximately 46% of the cells being transfected.

The results shown in FIG. 2 demonstrate the transfection reagents of the present invention are improved over current transfection reagents both in terms of percent of cells transfected and in percentage of cell death.

### Anion Exchange: TMTPS-Chloride Formation

### EXAMPLE 6

An aqueous solution of TMTPS-Iodide salt was passed through an anion exchange column containing Dowex® 1×8-200 resin in the chloride form. The resulting solution is then dried under vacuum to give the TMTPS-Chloride salt. This chloride salt is then used to formulate with neutral lipids as described in example 2.

Certain of the compounds of this invention may be insufficiently soluble in physiological media to employ for delivery and transfection methods. Those of ordinary skill in the art will appreciate that there are a variety of techniques available in the art to enhance solubility of such compounds in aqueous media. Such methods are readily applicable without undue experimentation to the compounds described herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms.

When a group of materials, compositions, components or compounds is disclosed herein, it is understood that all individual members of those groups and all subgroups thereof are disclosed separately. When a Markush group or other grouping is used herein, all individual members of the group and all combinations and subcombinations possible of the group are intended to be individually included in the disclosure. Every formulation or combination of components described or exemplified herein can be used to practice the invention, unless otherwise stated. Whenever a range is given in the specification, for example, a temperature range, a time range, or a composition range, all intermediate ranges and subranges, as well as all individual values included in the ranges given are intended to be included in the disclosure. In the disclosure and the claims, “and/or” means additionally or alternatively. Moreover, any use of a term in the singular also encompasses plural forms.

One of ordinary skill in the art will appreciate that starting materials, reagents, purification methods, materials, substrates, device elements, analytical methods, assay methods, mixtures and combinations of components other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention illustratively described herein suitably may be practiced in the absence of any element or elements, limitation or limitations which is not specifically disclosed herein.

All patents and publications mentioned in the specification are indicative of the levels of skill of those skilled in the art to which the invention pertains. References cited herein are incorporated by reference herein in their entirety to indicate the state of the art as of their filing date and it is intended that this information can be employed herein, if needed, to exclude specific embodiments that are in the prior art. For example, when a compound is claimed, it should be understood that compounds known in the art including the compounds disclosed in the references disclosed herein are not intended to be included in the claim.

All publications referred to herein are incorporated herein to the extent not inconsistent herewith. Some references provided herein are incorporated by reference to provide details of additional uses of the invention.

1. A liposomal composition comprising:
(a) one or more cationic lipids, or mixtures thereof, where the cationic lipids have the formula: 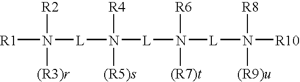
or salts or polycations thereof, where r, s, t and u independent of one other are 0 or 1 to indicate the presence or absence of the individual group, wherein when N is tetravalent it is positively charged, and wherein at least one of r, s, t or u is 1;
where L is a divalent organic radical independently selected from the group consisting of an alkylene group having from 1 to about 8 carbon atoms, wherein one or more non-neighboring —CH₂— groups can be replaced with an O or S atom, and wherein one or more carbon atoms of the group can be substituted with an OH, SH, SR or OR group, where R is an alkylene group having from 1 to about 6 carbon atoms;
where R1-R10 are independently selected from the group consisting of hydrogen and a C₁₋₂₂ alkyl, C₂₋₂₂ alkenyl, C₂₋₂₂ alkynyl and C₁₋₂₂ aryl, optionally substituted with one or more of an alcohol, aminoalcohol, hydroxyl, amine, carbohydrate, ether, polyether, amide, polyamide, ester, mercaptan, urea, thiourea, heterocyclic group, or heterocyclic aromatic group or carbon atoms and wherein one or more non-neighboring —CH₂— groups can be optionally replaced with an O or S atom;
where between 2 and 4 groups of R1-R10 are selected from the group consisting of a C₈₋₂₂ alkyl, C₈₋₂₂ alkenyl, C₈₋₂₂ alkynyl and C₈₋₂₂ aryl, optionally substituted with one or more of an alcohol, aminoalcohol, hydroxyl, amine, carbohydrate, ether, polyether, amide, polyamide, ester, mercaptan, urea, thiourea, heterocyclic group, or heterocyclic aromatic group and wherein one or more non-neighboring —CH₂— groups can be optionally replaced with an O or S atom;
where between 0 and 6 groups of R1-R10 are independently selected from the group consisting of a C₁₋₆ alkyl, C₂₋₆ alkenyl and C₂₋₆ alkynyl;
where not all of R1, R2, R3, R8, R9 and R10 are hydrogens; and
where when the cationic lipids are cationic lipid salts, the cationic lipid salts comprise an anion; and
(b) one or more neutral lipids, wherein the molar ratio of the one or more cationic lipids to the one or more neutral lipids in the liposomal composition is between 1:0.8 and 1:3.0.

2. (canceled)

3. (canceled)

4. The liposomal composition of claim 1, where each of R2, R4, R6 and R8 are a C₁₆ alkyl.

5. The liposomal composition of claim 1, where between 2 and 6 groups of R1-R10 are independently selected from the group consisting of a C₁₋₆ alkyl, C₂₋₆ alkenyl and C₂₋₆ alkynyl.

6. The liposomal composition of claim 1, where between 4 and 6 groups of R1-R10 are CH₃.

7. The liposomal composition of claim 1, where R3, R5, R7 and R9 are CH₃.

8. (canceled)

9. The liposomal composition of claim 1, where R1, R3, R9 and R10 are selected from the group consisting of hydrogen and an alkyl having 1 to 3 carbon atoms; R2, R4, R6 and R8 are selected from the group consisting of an alkyl having 12 to 20 carbon atoms, and R5 and R7 are CH3.

10. The liposomal composition of claim 1, where L is selected from the group consisting of an alkyl having 2 to 4 carbon atoms; R1, R5, R7 and R10 are CH3; R3 and R9 are hydrogen; and R2, R4, R6 and R8 are an alkyl having 16 carbon atoms.

11. The liposomal composition of claim 1, where the molar ratio of the one or more cationic lipids to the one or more neutral lipids in the liposomal composition is between 1:1.6 and 1:2.3.

12. The liposomal composition of claim 1, where the molar ratio of the one or more cationic lipids and one or more neutral lipids is between 1:1.6 and 1:1.9.

13. The liposomal composition of claim 1, wherein the molar ratio of the one or more cationic lipids to the one or more neutral lipids in the liposomal composition is 1:1.5 or above.

14. (canceled)

15. The liposomal composition of claim 14, wherein the anion is halide.

16. The liposomal composition of claim 15, wherein the anion is chloride.

17. The liposomal composition of claim 1, wherein at least 90% of the liposomal composition is a population of liposomal particles having a particle size of between 120 nm and 800 nm.

18. A method for introducing a nucleic acid into one or more cells comprising the steps:
(a) forming a liposomal composition comprising one or more cationic lipids, or mixtures thereof, where the cationic lipids have the formula: 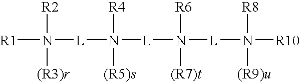
or salts or polycations thereof,
where r, s, t and u, independently of one other, are 0 or 1 to indicate the presence or absence of the individual group, wherein when N is tetravalent it is positively charged, and wherein at least one of r, s, t or u is 1;
where L is a divalent organic radical independently selected from the group consisting of an alkylene group having from 1 to about 8 carbon atoms, wherein one or more non-neighboring —CH₂— groups can optionally be replaced with an O or S atom, and wherein one or more carbon atoms of the group can be substituted with an OH, SH, SR or OR group, where R is an alkylene group having from 1 to about 6 carbon atoms;
where R1-R10 are independently selected from the group consisting of hydrogen and a C₁₋₂₂ alkyl, C₂₋₂₂ alkenyl, C₂₋₂₂ alkynyl and C₁₋₂₂ aryl, optionally substituted with one or more of an alcohol, aminoalcohol, hydroxyl, amine, carbohydrate, ether, polyether, amide, polyamide, ester, mercaptan, urea, thiourea, heterocyclic group, or heterocyclic aromatic group or carbon atoms and wherein one or more non-neighboring —CH₂— groups can be optionally replaced with an O or S atom;
where between 2 and 4 groups of R1-R10 are selected from the group consisting of a C₈₋₂₂ alkyl, C₈₋₂₂ alkenyl, C₈₋₂₂ alkynyl and C₈₋₂₂ aryl, optionally substituted with one or more of an alcohol, aminoalcohol, hydroxyl, amine, carbohydrate, ether, polyether, amide, polyamide, ester, mercaptan, urea, thiourea, heterocyclic group, or heterocyclic aromatic group and wherein one or more non-neighboring —CH₂— groups can be optionally replaced with an O or S atom;
where between 0 and 6 groups of R1-R10 are independently selected from the group consisting of a C₁₋₆ alkyl, C₂₋₆ alkenyl and C₂₋₆ alkynyl;
where not all of R1, R2, R3, R8, R9 and R10 are hydrogens;
where, when the cationic lipids are cationic lipid salts, the cationic lipid salts comprise an anion;
and one or more neutral lipids, wherein the molar ratio of the one or more cationic lipids to the one or more neutral lipids in the liposomal composition is between 1:0.8 and 1:3.0;
(b) combining the liposomal composition with the nucleic acid to form a liposome-macromolecule complex;
(c) contacting one or more cells with the liposome-nucleic acid complex to thereby introduce the nucleic acid into the one or more cells.

19. (canceled)

20. (canceled)

21. The method of claim 18, where L is selected from the group consisting of an alkyl having 2 to 4 carbon atoms; R1, R5, R7 and R10 are CH3; R3 and R9 are hydrogen; and R2, R4, R6 and R8 are an alkyl having 16 carbon atoms.

22. (canceled)

23. (canceled)

24. (canceled)

25. The method of claim 18, where the one or more neutral lipids comprises cholesterol or DOPE or a mixture thereof.

26. The method of claim 18, where the one or more cells are selected from the group consisting of CHO, A549, NIH3T3 and HeLa cells.

27. The method of claim 18, where the one or more cells are non-adherent cells.

28. The method of claim 27, where the one or more cells are non-adherent CHO cells.

29. The method of claim 27, where the one or more cells are contained in a non-adherent suspension cell culture.

30. The method of claim 29, where the nucleic acid is DNA.

31. The method of claim 29, where the nucleic acid is RNA.

32. The method of claim 29, where the nucleic acid is a vector.

33. The method of claim 29, where the nucleic acid is an expression vector.

34. The method of claim 33, further comprising incubating the one or more cells after the nucleic acid is introduced into the one or more cells, wherein a protein is expressed from the expression vector during the incubating step.

35. The method of claim 34, further comprising isolating the protein.

36. (canceled)

37. (canceled)

38. (canceled)

39. The method of claim 18, where the liposomal composition is formed using extrusion, sonication or microfluidization.

40. (canceled)

41. (canceled)

42. (canceled)

43. The method of claim 18, wherein the anion is halide.

44. The method of claim 43, wherein the anion is chloride.

45. The method of claim 18, wherein at least 90% of the liposomal composition is a population of liposomal particles having a particle size of between 120 nm and 800 nm.
